(19) United States
(12) Patent Application Publication
(10) Pub. No.: US 2013/0072513 A1
(43) Pub. Date: Mar. 21, 2013

(54) COMBINATIONS OF BERBERINE, ARTEMISININ, Loperamide AND THEIR DERIVATIVES TO TREAT MALARIA, DIARRHEA, TRAVELERS' DIARRHEA, DYSENTERY, DENGUE FEVER, PARASITES, CHOLERA AND VIRUSES

(71) Applicant: John Colman, Margate, FL (US)

(72) Inventor: John Colman, Margate, FL (US)

(73) Assignees: James Spencer, West Palm Beach, FL (US); Kirk Seubert, Bayside, NY (US)

(21) Appl. No.: 13/024,161

(22) Filed: Feb. 9, 2011

Related U.S. Application Data
(63) Continuation-in-part of application No. 12/428,465, filed on Apr. 22, 2009.
Application No. 12/428,465 is a division of application No. 13/024,151, filed on Feb. 9, 2011.

(51) Int. Cl. A61K 31/4741; A61P 31/04; A61P 33/10; A61P 31/18; A61P 31/20; A61P 31/14; A61P 33/06; A61P 1/12

(52) U.S. Cl. 514/280

(57) ABSTRACT

This invention relates to compositions and methods of combining berberine, artemisinin and loperamide or their derivatives in a therapeutic product for mammals suffering from malaria, diarrhea, travelers' diarrhea, dysentery, dengue fever, parasites, cholera and viruses by administration of a therapeutically effective amount of the composition.

This Application Claims Priority of Non-Provisional application Ser. No. 12/428,465 filed Apr. 22, 2009 and Non-Provisional application Ser. No. 13/024,151 filed on Feb. 9, 2011.

### FIELD OF INVENTION

This invention relates to compositions and methods that treat bacterial, viral, or parasitic infection including malaria and gastrointestinal infections presenting as acute diarrhea.

Malaria

This once widespread disease is now mainly confined to Africa, Asia and Latin America but still affects approximately 120 million people worldwide and results in over 1 million deaths per year. Controlling malaria in these countries is difficult because of inadequate health structures and poor socioeconomic conditions and the last few years have seen an increase in resistance to the drugs normally used to combat the parasite that causes the disease. Malaria is caused by four species of the protozoan parasites of the genus Plasmodium: Plasmodium falciparum, Plasmodium vivax Plasmodium ovale, and Plasmodium malaria. P. falciparum is the most widespread and dangerous of the four and if untreated can lead to fatal cerebral malaria.

Malaria parasites are transmitted from one person to another by the female anopheline mosquito that feeds on human flesh, as the males feed only on plant juices and do not transmit the disease. There are about 380 species of anopheline mosquito, but only about 60 transmit the parasite Like all other mosquitos, the anophelines breed in water and each species has its preferred breeding grounds, feeding patterns and resting place. Their sensitivity to insecticides is highly variable between species.

The plasmodium develops in the gut of the mosquito and is passed on in the saliva of an infected insect each time it takes a new blood meal. The parasites are then carried by the blood in the victim's liver where they invade the cells and multiply. After 9-16 days they return to the blood and penetrate the red cells, where they multiply again, progressively break down the red cells and induce fever and anaemia in the infected individual. In cerebral malaria, the infected red cells obstruct the blood vessels in the brain and other vital organs leading to the death of the patient.

Malaria is diagnosed by the clinical symptoms and microscopic examination of the blood and can usually be cured by antimalarial drugs. The symptoms, fever, shivering, pain in the joints and headache quickly disappear once the parasite is destroyed. In certain regions, however, the parasites have developed resistance to certain antimalarial drugs, particularly chloroquine. Patients in these areas require treatment with other more expensive drugs and hospitalization of Cases of severe disease including cerebral malaria. In endemic regions, where transmission is high, people are continuously infected.

Systematic control of malaria started after the discovery of the malaria parasite by Laveran in 1889, for which he received the 1907 Nobel Prize for medicine, and the demonstration by Ross in 1897 that the mosquito was the vector of malaria. These discoveries quickly led to control strategies and with the invention of DDT during the World War II, and the notion of global eradication of the disease. Effective and inexpensive drugs of the chloroquine group were also synthesized around this time. In 1969 it was recognized that global eradication of malaria was unlikely ever to be achieved and ongoing control programs remain essential in endemic areas. Malaria is currently endemic in 91 countries with small pockets of transmission occurring in an additional eight countries. Plasmodium falciparum remains the predominant parasite. More than 120 million clinical cases and over 1 million deaths occur in the world each year.

Children remain the most highly vulnerable to death from malaria followed by pregnant women whose natural immunity is reduced. Eighty percent of the cases occur in tropical Africa, where malaria accounts for 10% to 30% of all hospital admissions and is responsible for 15% to 25% of all deaths of children under the age of five. Around 800,000 children under the age of five die from malaria every year, making this disease one of the major causes of infant and juvenile mortality and a substantial number of miscarriages and low birth weight babies.

Malaria thus has social consequences and is a heavy burden on economic development. It is estimated that a single bout of malaria costs a sum equivalent to over 10 working days in Africa. The cost of treatment is between eight cents (US $ 0.08) and five dollars and thirty cents (US $5.30) according to the type of drugs prescribed as determined by drug resistance in the locality. In 1987, the total “cost” of malaria—health care, treatment, lost production, etc. was estimated to be eight hundred million United States dollars (US $800 million) for tropical Africa and this figure is currently estimated to be more than US $1,800 million.

The geographic distribution of malaria is in tropical and subtropical countries and its prevalence varies greatly from country to country and within the countries themselves. In 1990, seventy-five percent (75%) of all recorded cases outside of Africa were concentrated in nine countries: India, Brazil, Afghanistan, Sri Lanka, Thailand, Indonesia, Vietnam, Cambodia and China. The incidence rates in South American and Carribean countries have also significantly increased. Traditional endemic zones where transmission had once been eradicated have suffered epidemics, increasing malaria's significance as a world health problem. These outbreaks are generally associated with deteriorating social and economic conditions, and the victims are travelers and underprivileged rural populations. Demographic, economic and political pressures compel entire populations of seasonal workers, nomadic tribes and farmers to leave malaria free areas and move into newly developed agricultural and urban areas in endemic zones. The vast majority of people are non-immune and at high risk of severe disease. Unfortunately, these population movements and the intensive urbanization are not always accompanied by adequate development of sanitation and health care. This frequently results in a recourse to self-administration of drugs, incomplete treatment, and parasitic resistance to previously effective drugs.

Infectious Diarrhea

Chronic diarrhea is usually related to functional disorders such as irritable bowel syndrome or inflammatory bowel disease and acute diarrhea is most often caused by bacterial, viral, or parasitic infection but may be caused by food intolerances to artificial sweeteners, lactose, and other food components. The more common bacterial causes of diarrhea include several types of bacteria consumed through contaminated food or water including Campylobacter, Salmonella, Shigella, and Escherichia coli (E. coli). Viral etiologies include rotavirus, Norwalk virus, cytomegalovirus, herpes simplex virus, and viral hepatitis. Parasites can enter the body through food or water and settle in the digestive system. Diarrhea may be caused by Giardia lamblia, Entamoeba histolytica, and Cryptosporidiu.

Enterotoxins

Certain forms of diarrhea, namely those caused by the production of enterotoxins by certain bacteria are often difficult to treat and can be life threatening. The use of normal antidiarrheal agents which merely are absorbents, such as kaolin, or agents that reduce intestinal muscle activity have little or no effect on this enterotoxin-caused diarrhea. Cholera is an acute infection of Vibrio cholerae (V. Cholerae) in man involving the entire small bowel, characterized by a debilitating diarrhea. Enterotoxigenic Escherichia coli are a major cause of diarrhea in neonatal food-producing animals and “travelers diarrhea” in man.

These enteric bacteria release enterotoxins released by these enteric bacteria reverse the transport of water and electrolytes in the epithelium of the small intestine, from absorption to secretion. V. cholerae enterotoxin and Escherichia coli LT enterotoxin stimulate adenylate cyclase causing increased formation of cAMP which, through a cascade of reactions, inhibits active absorption and stimulates active secretion of water and sodium, chloride and bicarbonate electrolytes. In contrast to these two enterotoxins, Escherichia coli ST enterotoxin stimulates guanylate cyclase, increases cGMP levels and causes diarrhea primarily by inhibiting active absorption of water and electrolyte.

Traveler's Diarrhea

Travelers' diarrhea (TD) is the most common traveler's illness, affecting 20%-50% of international travelers, an estimated 10 million persons, per year. The onset of TD usually occurs within the first week of travel but may occur at any time while traveling, and even after returning home. Risk is a factor of destination and is highest in the developing countries of Latin America, Africa, the Middle East, and Asia. Persons at particular high-risk include young adults, immunosuppressed persons, persons with inflammatory-bowel disease or diabetes, and persons taking H-2 blockers or antacids. Incidence rates are similar for men and women. The primary source of infection is ingestion of fecally contaminated food or water. TD begins abruptly with stool of increased frequency, volume, and weight and altered consistency, typically, four to five loose or watery bowel movements each day. Other commonly associated symptoms are nausea, vomiting, abdominal cramping, bloating, fever, urgency, and malaise.

TD is rarely life-threatening. Ninety percent (90%) of cases resolve naturally within 1 week, and ninety eight percent (98%) resolve within 1 month. Infectious bacterial, viral and parasitic enteric pathogens are the primary cause of TD and bacterial enteropathogens, most commonly enterotoxigenic E. Coli (ETEC), are responsible for approximately 80% of cases. ETEC produces watery diarrhea with associated cramps and low-grade or no fever.

Dysentery

Dysentery is a term used for diarrhea when there is evidence of pathogenic invasion of the intestinal wall, causing pus, mucus, and blood to appear in the stool and frequently fever and abdominal cramps. There is no bright-line distinctions between diarrhea and dysentery. Attempts to define their differences in medical and travel advice books is misplaced as treatments are often the same.

Dengue Fever

In 2005, dengue (DF) was the most important mosquito-borne viral disease affecting humans; its global distribution is comparable to that of malaria, and an estimated 2.5 billion people live in areas at risk for epidemic transmission (FIG. 4). Each year, tens of millions of cases of DF occur and, depending on the year, up to hundreds of thousands of cases of dengue hemorrhagic fever (DHF). The case fatality rate of DHF in most countries is about 5%, mostly in children and young adults, but proper treatment can reduced it to less than 1%. There is a small risk for dengue outbreaks in the continental United States. Two competent mosquito vectors, Ae. aegypti and Aedes albopictus, are present and, under certain circumstances, could transmit dengue viruses. This type of transmission has been detected six times in the last 25 years in south Texas (1980 -2004) and has been associated with dengue epidemics in northern Mexico by Aedes aegypti and in Hawaii (2001-02) due to Ae. albopictus. Moreover, numerous viruses are introduced annually by travelers returning from tropical areas where dengue viruses are endemic. From 1977 to 2004, a total of 3,806 suspected cases of imported dengue were reported in the United States. Although some specimens collected were not adequate for laboratory diagnosis, 864 (23%) cases were confirmed as dengue and many more cases are likely to be unreported. Surveillance in the United States is passive and relies on physicians to recognize the disease, inquire about the patient's travel history, obtain proper diagnostic samples, and report the case. These data suggest that southern and southeastern states, where Ae. aegypti is found, are at risk for dengue transmission and sporadic outbreaks. Although travel-associated dengue and limited outbreaks do occur in the continental United States, most dengue cases in US citizens occur as endemic transmission among residents in some of the US territories. The Center for Disease Control (CDC) conducts laboratory-based passive surveillance in Puerto Rico in collaboration with the Puerto Rico Department of Health.

The reasons for the dramatic global emergence of DF/DHF as a major public health problem are complex and not well understood but several important factors can be identified. First, major global demographic changes have occurred, the most important of which have been uncontrolled urbanization and concurrent population growth. These demographic changes have resulted in substandard housing and inadequate water, sewer, and waste management systems, all of which increase Ae. aegypti population densities and facilitate transmission of Ae. aegypti-borne disease. In most countries the public health infrastructure has deteriorated. Limited financial and human resources and competing priorities have resulted in a “crisis mentality” with emphasis on implementing so-called emergency control methods in response to epidemics rather than on developing programs to prevent epidemic transmission. This approach has been particularly detrimental to dengue control because, in most countries, surveillance is, as in the U.S., passive; the system to detect increased transmission normally relies on reports by local physicians who often do not consider dengue in their differential diagnoses. An epidemic has often reached or passed its peak before it is recognized. Increased travel by airplane provides the ideal mechanism for infected human transport of dengue viruses between population centers of the tropics, resulting in a frequent exchange of dengue viruses and other pathogens.

Lastly, effective mosquito control is virtually nonexistent in most dengue-endemic countries. Considerable emphasis in the past has been placed on ultra-low-volume insecticide space sprays for adult mosquito control, a relatively ineffective approach for controlling Ae. aegypti. No dengue vaccine is yet available. While attenuated candidate vaccine viruses have been developed recently, efficacy trials in human volunteers have not been initiated. Research is also being conducted to develop second-generation recombinant vaccine viruses. Therefore, an effective dengue vaccine for public use will not be available for 5 to 10 years. Prospects for reversing the recent trend of increased epidemics and geographic expansion of dengue are not promising. New dengue virus strains and serotypes will likely continue to be introduced into many areas where the population densities of Ae. aegypti are at high levels.

Artemisinin

Artemisinin and its Chemical Derivatives Artemisinin, or (ginghaosu), a clinically useful antimalarial agent which was isolated from the plant Artemisia annua, is an unusual sesquiterpene lactone containing an epidioxide function. Dihydroartemisinin, obtained by sodium borohydride reduction of artemisinin, has been reported as more therapeutically active than its parent compound. Neither artemisinin nor dihydroartemisinin exhibit cross-resistance to chloroquine and both were proven efficacious against cerebral malaria in man. Artesunate, or sodium artesunate, the sodium salt of the succinic acid half ester derivative of dihydro-artemisinin, is water soluble and can be administered intravenously, making the compound particularly useful in the treatment of cerebral malaria where rapid administration is critical. Artesunate, however, has been shown in human clinical trials to be as effective as injectable artesunate to treat uncomplicated malaria. Alin, M H, et al, 53(6) Am J Trop Med Hyg 639-45 (1995); .Luxembuger, C, et al, 53(5) Am. J. Trop Med Hyg 522-5 (1995).

Artemisinin was first isolated by the Chinese in 1972, and was soon discovered to be a fast acting, safe and effective drug against chloroquine-resistant and sensitive strains of Plasmodium falciparum, as well as cerebral malaria. No side effects, common to many synthetic anti-malarials, have been reported by the Chinese during the past 35 years of clinical use of artemisinin or artesunate. One disadvantage of artemisinin itself is that the compound is only sparingly soluble in either water or oils and thus not readily absorbable by the gastrointestinal tract. There was a long-felt need for a more ideal drug or a combination of drugs with enhanced antimalarial activity and improved physical and bioavailability properties such as artesunate for more effective oral treatment of malaria and the treatment of chloroquine-resistant malaria.

A search of the USPTO under artemisinin and malaria listed 109 patents relating to artemisinin and its chemical derivatives, novel delivery systems, treatment of a variety of protozoal and viral diseases, such as toxoplasmosis, HIV, cancer treatments, treatment of infectious diseases, and antimicrobial compositions. Romero, M R, et al 68(2) Antiviral Res 75-83 (2005); Jiao, Y, et al 28(7) Acta Pharmacol Sin 1045-56 (2007); Shoeb, H A, et al, 2(6) J Chemother 362-7 (1990); Wang, J, et al, 50(7) Antimicrob agents Chemother 2420-7 (2006).

A search of the USPTO database for dihydroartemisinin and malaria listed 46 patents for treatment of a wide variety of diseases that primarily teach new analogs of artemisinin and dihydroartemisinin and their uses.

Berberine

Berberine (5,6-Dihydro-9,10-dimethoxybenzo[g]-1,3-benzodioxolo[5,6-a]quinolizinium) is an alkaloid present in various species of Berberis and several other plant families. Oral berberine has both anti-secretory and antimicrobial properties and is nontoxic at high oral doses.

For many centuries, berberine extract from plants has been used by traditional practitioners in both India and China to manage a variety of medical conditions, including acute diarrhea. Berberine shows in vitro activity against the protozoa Trichomonas vaginalis, Giardia lamblia, Entamoeba histolytica, several of the protozoal strains which cause leishmaniasis, as well as several types of fungi, bacteria, viruses, and the human immunodeficiency virus (HIV).

Rabbani, G H. 43(2) Dan Med Bull 173-85 (1996); Soffar, S A, et al, 31(3) J. Egypt Soc Parasitol 893-904 (2001); Hawrelak, J, 8(2) Altern Med Rev 129-42 (2003); Vennerstrom, J L, et al, 34(5) Antimoicrob Agents Chemother 918-21 (1990); Kaneda, Y, et al, 85(4) Ann Trop Med Parasitol. 417-25 (1991); Tauk, L, et al, 78(2) Fitoterapia 159-61 (2007); Sun, D., Courtney, H S, Beachey, E H, 32(9) Antimicrob Agents Chemother 1370-4 (1988); Gudima, S O November-December (6) Mol Biol 1308-14 (1994).

Berberine, purified as a hydrochloride, sulfate, or tannate salt, has been used clinically to treat bacterial, fungal and some protozoal infections. Orally administered berberine has been shown to be a safe and effective agent against acute diarrhea, such as that caused by the protozoal pathogen G. lamblia, Kaneda, Y, et al, 85(4) Ann Trop Med Parasitol. 417-25 (1991), Escherichia coli and Vibrio cholerae toxins.

Healing of sores caused by cutaneous species of leishmanial parasites has been effected by intradermal administration of berberine. Other uses of various berberine compounds are disclosed in Maroko, U.S. Pat. Nos. 5,153,178, 4,980,344, 4,749,708, and 4,761,417, each of which is incorporated herein by reference.

Berberine has shown in vitro activity against telomerase activity of the malaria plasmodium fulciparum during its erythrocyte cycle. Sriwilaijareon, N, et al, 51(1) Parasitol Int 99-103 (2002). As a major active constituent of a Vietnamese medicinal plant, berberine was found to have antiplasmodial activity by inhibiting the growth of a Plasmodium falciparium strain FCR-3 with EC(50) values of less than 10 micrograms per ml added to cell culture. Tran, Q L, et al, 86(2-3) J. Ethnopharmacol 249-52 (2003). In a human trial of 215 patients with chloroquine resistant malaria, berberine in combination with pyrimethamine, showed a clearance rate of asexual parasitaemia of 74.4%, higher than a tetracycline only group (67.2%) or a cotrimoxazole only group with a clearance rate of only 47.8%. Sheng, W D, et al, 74(5) East AFR Med J 283-4 (1997).

A search of the USPTO data base for berberine and malaria lists 13 patents that do not mention the treatment of malaria with berberine specifically. Prior to this invention, berberine has not been used in combination with artemisinin or its derivatives for the treatment of malaria in humans. Berberine and arteminisin have not been combined to treat multiple etiological agents, such as malaria, diarrhea, dysentery and other bacteria or parasites simultaneously.

Berberine and arteminisin have not previously been combined in an acute dosage regimine, nor have berberine and arteminisin been previously been combined in a blister pack to make dosing compliance simple for patients or travelers. A search of the USPTO database for berberine and diarrhea lists 25 patents, new compositions and uses for treating diverse conditions from diarrhea, circulation, Alzheimer's, depression, diaper rash, scar tissue, cancer, termite eradication and the like. No patents were revealed by searches of the USPTO database for berberine and dengue fever or travelers diarrhea and kit.

Loperamide

When a traveler is suffering from diarrhea, it is often necessary to control the symptoms of the disease while the multiple etiological agents, such as malaria, diarrhea, dysentery and other bacteria or parasites are treated, in this invention with Berberine and arteminisin have not been combined to treat. Loperamide, 4-[4-(4-chlorophenyl)-4-hydroxypiperidin-1-yl]-N,N-dimethyl-2,2-diphenylbutanamide, has well-established clinical history of use in the sympomatic control of diarrhea. It is frequently used in medicine as a hydrochloride salt, oxide, or N-oxide form.

Loperamide is considered to be an opoid agonist, Elkiweri, M S, et al, 108 Anesth Analg 149-159 (2009), though its effects on the central nervous system are not as potent as other opoids. Peripheral injection of mu-opioid agonist loperamide virtually did not modify the central compartments of the opioid system under conditions of morphine treatment. Methylnaloxone and loperamide partially prevented the development of morphine analgesia tolerance. Sudakov S K, Trigub M M, 146(6) Bull Exp Biol Med 663-6 (2008).

Japanese researchers have shown that the mechanism of action for loperamide is an effect on Peptide YY (PYY) that is produced in the lower gastrointestinal tract and has antisecretory effects in the colon and inhibition of gastrointestinal motility. Loperamide administration to rats resulted in multiple changes in plasma and intestinal mucosa PYY concentrations, along with an improvement in the diarrhea. Our research showed that the endocrine hormone PYY is involved in the onset of diarrhea, the course of the condition, and the manifestation of medicinal effects in the lower intestine. Hirotani Y, et al, 128(9) Yakugaku Zasshi 1311-6 (2008).

Several inventors hold patents relating to the use of loperamide in diarrhea, i.e., U.S. Pat. No. 6,869,602, to Ryu, et al, for diarrhea, but its combination with either berberine or artemisinin has not been heretofore disclosed. It has been used in the form of loperamide hydrochloride with saccharide selected from the group consisting of sucrose, fructose, glucose, sorbitol, xylitol, mannitol, and mixtures thereof, said saccharide being present in an amount of from 9 to about 98 wt. % of said composition and from 3,000 times to 20,000 times the weight of said loperamide hydrochloride. U.S. Pat. No. 5,182,112, to Kurazumi, et al.

### SUMMARY OF INVENTION

This inventor has discovered that a composition comprising berberine, artemesinin and loperamide or their derivatives may be used to effectively treat multiple pathogens affecting humans simultaneously in a therapeutic product. Travelers and citizens of third world countries are often simultaneously exposed to, and afflicted with, a variety of pathogenic substances. An objective of this invention is to provide a single therapeutic agent designed to treat a single pathogen, such as the malaria parasite can be life-saving, but concomitant administration of other drugs to treat diarrhea, dysentery, other parasites, E. coli, Vibrio coli, fungal infections, viruses, or dengue fever may be necessary. Proper treatment is complicated by the inability of the physician or practitioner to diagnose and separately identify other pathogens, particularly in third world hospitals with limited testing abilities. The prognosis for either malaria, dengue fever, fungal infections, E. coli, Vibrio coli, or other viral infections becomes less clear when multiple pathogens are present.

Treatment is further complicated by the need to ingest multiple drugs in a complex regimen where the patient can be acutely sick or delirious and have no attending nurse or guardian present. There is a long-felt need to combine an extremely broad spectrum antibacterial, antimicrobial, antiviral, antiparasitic and antifungal agent such as berberine with its long, well-established use to treat intestinal pathogens with artemisinin or its derivatives, as effective agents for treating malaria, viruses and other blood parasites. A second objective of the invention is to provide the composition in dosage formulations that include, but are not limited to, spray bottles, fast melt pill format, bursts, gel format, adhesive bandages, skin patches, gelcaps, softgels, gelatin capsules, vegetarian capsules, hard shell gelatin capsules, injections, intravenous solutions, topical creams, topical ointments, suppositories, or sublingual methods of administration known to those versed in the art.

A third objective of the invention is to provide a compact traveler's kit consisting of a blister pack with several day's separate doses of berberine, artemisinin and loperamide in one pill or capsule and simplify compliance of afflicted persons. The method of packaging of the travelers' kit of this invention includes, but is not limited to: blister packs, zip lock packs, standup pouches, foil pouches, boxes, jars, bottles, single dose packets, one a day packs, two day packs, three day packs, and the like. Kits may contain dosages needed for any number of days, from four to thirty days, sixty days, or ninety days.

The oral dosage ranges of artemisinin or its derivatives may be from about 1 mg to about 1,500 mg per dose of artemisinin, artesunate, sodium artesunate, dihydroartemisinin or any of the artemisinin analogs described by this inventor, administered one to three times daily to a human. The oral dosage ranges are more specifically about 20 mg to about 250 mg per dose of artesunate per human taken one to three times daily, more specifically about 40 mg to about 100 mg per dose of artesunate per human taken one to three times daily and most specifically about 50 mg per dose of artesunate per human taken twice daily.

The oral dosage range of berberine, its salts or derivatives may be about 50 mg to about 1,500 mg in a single human dose administered two to three times daily, not to exceed about 4,500 mg per day. The oral dosages ranges are more specifically about 100 mg to about 1,000 mg in a single human dose not to exceed 3,000 mg per day administered one to three times daily, more specifically a single human dose of berberine at about 200 mg to about 500 mg taken one to three times daily and most specifically a single dose of berberine at about 200 mg taken twice daily.

The oral dosage of loperamide or its derivatives may be from about 0.1 mg to about 200 mg in a single human dose administered two to three times daily, not to exceed 500 mg per day. The oral dosages ranges are more specifically about 5 mg to about 10 mg in a single human does not to exceed 20 mg per day administered one to three times daily, and most specifically about 2 mg per dose of loperamide per human taken twice daily.

### DETAILED DESCRIPTION OF INVENTION

In one embodiment, this invention teaches a composition for the treatment of infectious diarrhea comprised of a therapeutically effective amount of berberine, with a blood anti-parasitic antimalarial agent, artemesinin, their pharmaceutically acceptable derivatives, salts, esters, chelates. Most specifically, the artemesinin derivative is the salt of the succinic acid half ester derivative of dihydro-artemisinin known as artesunate.

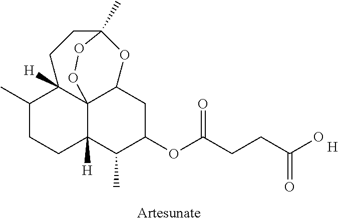

In this invention, the term berberine includes, but is not limited to, berberine alkaloid, berberine base, berberine hydrochloride, berberine, berberrubine, coreximine, tetrahydropalmatine, jatrorrhizine, 13-hydroxyberberine chloride, coralyne, coralyne chloride, 7,8-dihydro-13-methylberberine, berberine acetone, 13-allylberberine, palmatine, 13-benzylberberine, tetrahydroberberine, tetrahydroprotoberberine 8-cyanodihydroberberine, dimeric protoberberine alkaloids, demethylated protoberberine alkaloids, quataternary protoberberine alkaloids, protoberberine and protoberberine alkaloids.

In this invention, the salts of berberine, include berberine hydrochloride, berberine chloride, berberine sulfate, berberine tannate and other salts known to those versed in the art. In this invention, plant extracts containing berberine at a concentration of greater than 3%, include, but are not limited to, the Berberis family, Berberis aristata, Berberis aquifolllium, Berberis vulgaris, Berberis aetensis, Coptis chinensis, Chelidonium majus (Ukrain), goldenseal (Hydrastis canadensis), Rhizoma coptidis, Phellodendron chinense, Aquilegia oxysepala, Cortex phellodendra, Huanglian Jiedu decoction, San-Huang-Xie-Xin-Tang, Xietianwu, Gegen Quinlian, and Shizhu.

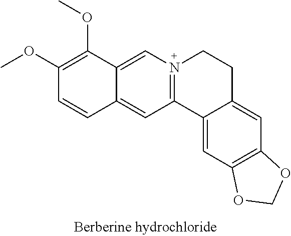

In this invention, the terms ‘artemisinin’ and ‘artesunate’ will be U.S. Pat. No. ______ to include artesunate, artemisinin, dihydroartemisinin, dihydroartemisinin hemisuccinate, dihydrodroartemisinin succinate, sodium artesunate, stabilized forms of artesunate, stabilized forms of sodium artesunate, dihydroartemisitene dimers (U.S. Pat. No. 7,098,242), amino-funtionalized 1,2,4-trioxanes (U.S. Pat. No. 7,071,226), artemisinin endoperoxides (U.S. Pat. No. 6,984,640), spiro and dispiro 1,2,4-trioxolane anti-malarials (U.S. Pat. No. 6,906,205), mixed steroidal 1,2,4,5-tetraoxane compounds (U.S. Pat. No. 6,906,098), arteether (U.S. Pat. No. 6,750,356), substituted 1,2,4-trioxanes (U.S. Pat. No. 6,737,438), Artemisia annua extracts (U.S. Pat. No. 6,685,972), artemether (U.S. Pat. No. 6,683,193, trioxane derivatives based on artemisinin (U.S. Pat. No. 6,649,647), trioxane dimer compounds (U.S. Pat. No. RE 38,117), conjugates of artelinic acid (U.S. Pat. No. 6,461,603.), arteethers from dihydroartemisinin (U.S. Pat. No. 6,346,631), artemisinine or artemisinene derivatives (U.S. Pat. No. 6,306,896.), C-10 carbon substituted artemisinin-like trioxane compounds (U.S. Pat. No. 6,160,004). Water-soluble trioxanes (U.S. Pat. No. 6,136,847), alpha arteether (U.S. Pat. No. 6,127,405.), artemisinin dimers (U.S. Pat. No. 5,856,351), (+)-deoxoarteminisinin and analogs of (+)-deoxoartemisinin (U.S. Pat. No. 5,225,562), and 10-substituted ether derivatives of dihydroartemisinin (U.S. Pat. No. 5,225,427).

Specifically, the berberine, its pharmaceutically acceptable derivatives, salts, chelates and esters, is present in an amount of about 50 mg to about 1500 mg; the artemisinin, its pharmaceutically acceptable derivatives, salts, chelates, and esters is present in an amount of about 1 mg to about 1500 mg; and the loperamide, its pharmaceutically acceptable derivatives, salts, chelates, and esters is persent in an amount of about 0.1 mg to about 200 mg. More specifically, the berberine, its pharmaceutically acceptable derivatives, salts, chelates and esters, is present in an amount of about 100 mg to about 1000 mg; the artemisinin, its pharmaceutically acceptable derivatives, salts, chelates, and esters is present in an amount of about 20 mg to about 250 mg and the loperamide, its pharmaceutically acceptable derivatives, salts, chelates, and esters is present in an amount of about 0.5 mg to about 10 mg. More specficially, the berberine, its pharmaceutically acceptable derivatives, salts, chelates and esters, is present in an amount of about 200 mg to about 500 mg; the artemisinin, its pharmaceutically acceptable derivatives, salts, chelates, and esters is present in an amount of about 40 mg to about 100 mg and the loperamide its pharmaceutically acceptable derivatives, salts, chelates, and esters is present in an amount of about 1 mg to about 3 mg. Most specifically, berberine its pharmaceutically acceptable derivatives, salts, chelates and esters, is present in an amount of about 200 mg; the artemisinin, its pharmaceutically acceptable derivatives, salts, chelates, and esters is present in an amount of about 50 mg and loperamide is present in an amount of about 2 mg.

The composition may be present in dosage formulations selected from the group consisting of spray bottles, fast melt pill format, bursts, gel format, adhesive bandages, skin patches, gelcaps, softgels, gelatin capsules, vegetarian capsules, hard shell gelatin capsules, injections, intravenous solutions, topical creams, topical ointments, suppositories, or sublingual formulations. It may further comprise a therapeutically effective amount mefloquine for the treatment of malaria.

In a specific embodiment of the invention, the berberine, artesunate and loperamide are packaged in a daily dispenser form with daily individual doses for a number of days of one day to ninety days, and the daily dispenser may be in the form of a travelers pack.

Also taught are methods for the treatment of travelers suffering from at least one of multiple parasitic, bacterial or viral infections simultaneously by the administration to a traveler of a therapeutically effective amount of the composition of berberine, artesunate and loperamide, their pharmaceutically acceptable derivatives, salts, esters, or chelates. The administration may be performed between one and three times per day, more specifically between two and three times per day.

The method of treatment of a traveler may be fore the treatment of malaria, and the malaria may be chloroquine resistant. The same method of treatment may be used to treat mammals suffering from dysentery, diarrhea, or cholera. In another embodiment of the invention, the method of treatment is of a virus in a mammal such as dengue fever, hepatitis B, West Nile virus, or human immunodeficiency virus (HIV). In the case of human immunodeficiency virus (HIV), the treatment may be adjunctive and the human immunodeficiency virus may be anti-retroviral resistant.

In yet another embodiment of the invention, the treatment is of intestinal parasites in a mammal, including a tapeworm or tapeworms, or toxoplasmosis.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A composition for the treatment of infectious diarrhea comprised of a therapeutically effective amount of berberine hydrochloride, with a blood anti-parasitic antimalarial agent, artemesinin and an anti-diarrheal agent, loperamide, their pharmaceutically acceptable derivatives, salts, esters, chelates.

2. The composition of claim 1 wherein said artemesinin derivative is the salt of the succinic acid half ester derivative of dihydro-artemisinin, artesunate.

3. A method of treatment a traveler suffering from at least one of multiple parasitic, bacterial or viral infections simultaneously by administration of a therapeutically effective amount of the composition of claim 2.

4. The method of claim 3 wherein said administration is performed between one and three times per day.

5. The method of claim 4 wherein said administration is performed between two and three times per day.

6. The method of claim 5 wherein said treatment is of malaria in a mammal in need thereof.

7. The method of treatment of claim 6 wherein said malaria is chloroquine resistant.

8. The method of claim 5 wherein said treatment is of dysentery in a mammal in need thereof.

9. The method of claim 5 wherein said treatment is of diarrhea in a mammal in need thereof.

10. The method of claim 5 wherein said treatment is of cholera in a mammal in need thereof.

11. The method of claim 5 wherein said treatment is of a virus in a mammal in need thereof.

12. The method of claim 11 wherein said treatment is of dengue fever in a mammal in need thereof.

13. The method of claim 11 wherein said treatment is of hepatitis B in a mammal in need thereof.

14. The method of claim 11 wherein said treatment is of human immunodeficiency virus (HIV) in a mammal in need thereof.

15. The method of claim 14 wherein the treatment for human immunodeficiency virus (HIV) in a mammal in need wherein said treatment is adjunctive.

16. The method of claim 14 wherein said human immunodeficiency virus is anti-retroviral resistant.

17. The method of claim 11 wherein said treatment is of West Nile virus in a mammal in need thereof.

18. The method of claim 5 wherein said treatment is of intestinal parasites in a mammal in need thereof.

19. The method of claim 18 wherein said treatment is of a tapeworm or tapeworms in a mammal in need thereof.
